SEQUENCE LISTING (23 sequences)

SEQ ID NO: 1 - protein, 317 aa, Homo sapiens
DTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLR

SEQ ID NO: 2 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 3 - protein, 322 aa, Homo sapiens
GGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNST

SEQ ID NO: 4 - protein, 129 aa, Homo sapiens [sequence omitted]

SEQ ID NO: 5 - protein, 111 aa, Homo sapiens
PVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 6 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 232 aa, synthetic construct
  REGION 1..232: note = Description of Artificial Sequence: Synthetic polypeptide
EPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 228 aa, synthetic construct
  REGION 1..228: note = Description of Artificial Sequence: Synthetic polypeptide
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 229 aa, synthetic construct
  REGION 1..229: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 585 aa, synthetic construct [sequence omitted]

SEQ ID NO: 11 - protein, 773 aa, synthetic construct
  REGION 1..773: note = Description of Artificial Sequence: Synthetic polypeptide
DTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRGGGGSGGGGSMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHGGGGSGGGGSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 20 aa, synthetic construct
  REGION 1..20: note = Description of Artificial Sequence: Synthetic peptide
  SITE 1..20: note = This sequence may encompass 1-10 "Gly Ser" repeating units
GSGSGSGSGSGSGSGSGSGS

SEQ ID NO: 13 - protein, 30 aa, synthetic construct
  REGION 1..30: note = Description of Artificial Sequence: Synthetic polypeptide
  SITE 1..30: note = This sequence may encompass 1-10 "Gly Gly Ser" repeating units
GGSGGSGGSGGSGGSGGSGGSGGSGGSGGS

SEQ ID NO: 14 - protein, 40 aa, synthetic construct
  REGION 1..40: note = Description of Artificial Sequence: Synthetic polypeptide
  SITE 1..40: note = This sequence may encompass 1-10 "Gly Gly Gly Ser" repeating units
GGGSGGGSGGGSGGGSGGGSGGGSGGGSGGGSGGGSGGGS

SEQ ID NO: 15 - protein, 40 aa, synthetic construct
  REGION 1..40: note = Description of Artificial Sequence: Synthetic polypeptide
  SITE 1..40: note = This sequence may encompass 1-10 "Gly Gly Ser Gly" repeating units
GGSGGGSGGGSGGGSGGGSGGGSGGGSGGGSGGGSGGGSG

SEQ ID NO: 16 - protein, 50 aa, synthetic construct
  REGION 1..50: note = Description of Artificial Sequence: Synthetic polypeptide
  SITE 1..50: note = This sequence may encompass 1-10 "Gly Gly Ser Gly Gly" repeating units
GGSGGGGSGGGGSGGGGSGGGGSGGGGSGGGGSGGGGSGGGGSGGGGSGG

SEQ ID NO: 17 - protein, 50 aa, synthetic construct
  REGION 1..50: note = Description of Artificial Sequence: Synthetic polypeptide
  SITE 1..50: note = This sequence may encompass 1-10 "Gly Gly Gly Gly Ser" repeating units
GGGGSGGGGSGGGGSGGGGSGGGGSGGGGSGGGGSGGGGSGGGGSGGGGS

SEQ ID NO: 18 - protein, 10 aa, synthetic construct
  REGION 1..10: note = Description of Artificial Sequence: Synthetic peptide
GGGGSGGGGS

SEQ ID NO: 19 - protein, 4 aa, synthetic construct
  REGION 1..4: note = Description of Artificial Sequence: Synthetic peptide
GSGS

SEQ ID NO: 20 - protein, 6 aa, synthetic construct
  REGION 1..6: note = Description of Artificial Sequence: Synthetic peptide
GGSGGS

SEQ ID NO: 21 - protein, 8 aa, synthetic construct
  REGION 1..8: note = Description of Artificial Sequence: Synthetic peptide
GGGSGGGS

SEQ ID NO: 22 - protein, 10 aa, synthetic construct
  REGION 1..10: note = Description of Artificial Sequence: Synthetic peptide
GGSGGGGSGG

SEQ ID NO: 23 - protein, 8 aa, synthetic construct
  REGION 1..8: note = Description of Artificial Sequence: Synthetic peptide
GGSGGGSG

(19) United States
(12) Patent Application Publication
(10) Pub. No.: US 2023/0242615 A1
(43) Pub. Date: Aug. 3, 2023

(54) FUSION POLYPEPTIDE

(71) Applicant: Beijing VDJBio Co., Ltd., Beijing (CN)

(72) Inventors: Ziqiang Li, Beijing (CN); Xinsheng Tian, Beijing (CN); Yiping Sun, Beijing (CN); Yiren Liu, Beijing (CN); Shaokui Li, Beijing (CN)

(21) Appl. No.: 18/064,054

(22) Filed: Dec. 9, 2022

Related U.S. Application Data
(63) Continuation of application No. PCT/CN2021/099832, filed on Jun. 11, 2021.
Application No. PCT/CN2021/099832 is a continuation of application No. PCT/CN2020/095875, filed on Jun. 12, 2020.

(51) Int. Cl. C07K 14/715; A61P 37/00

(52) U.S. Cl. CPC C07K 14/7155; A61P 37/00; C07K 2319/30; C07K 2319/31

(57) ABSTRACT

Provided is a fusion polypeptide as an antagonist for IL4 and IL13. Also provided is a pharmaceutical composition comprising the fusion polypeptide and a pharmaceutically acceptable excipient.

### CROSS-REFERENCE

This application is a continuation application of International Patent Application No. PCT/CN2021/099832, filed Jun. 11, 2021, which claims the benefit of PCT International Application No. PCT/CN2020/095875, filed Jun. 12, 2020, each of which application is incorporated herein by reference in its entirety.

### REFERENCE TO A SEQUENCE LISTING

The instant application contains a Sequence Listing which has been submitted electronically in XML, format and is hereby incorporated by reference in its entirety. Said XML, copy, created on Dec. 9, 2022, is named 46198_703_301_SL.xml and is 34,021 bytes in size.

### BACKGROUND OF THE INVENTION

Interleukin 4 (IL-4, IL4) is a cytokine produced mostly by mast cells, basophils, a subset of activated T cells, eosinophils and neutrophils. It has been considered as one of the most powerful cytokines in regulating immune system. The receptor for interleukin-4 is known as the IL4Rα. This receptor exists in different complexes throughout the body. Type I IL4 receptor complex is formed by IL4Rα subunit and IL-2Rγc, which is found in lymphocytes and myeloid cells. Type II IL4 receptor complex is formed by IL4Rα subunit and IL13Rα1, which has been shown to express in myeloid cells and all non-hematopoietic cells. These type II receptor is able to bind both IL4 and IL13, two cytokines with closely related biological functions.

Interleukin 13 (IL-13, IL13) is a cytokine which partially shares the signaling pathways with IL4 due to the utilization of a common receptor system, i.e. type II IL4 receptor complex. Initially, the ligand IL4 and IL13 bind to IL4Rα chain and IL13Rα1, respectively, then a secondary chain of IL13Rα1 and IL4Rα will also join to form the complete type II IL4 receptor complex which further activate the JAK-STAT signaling pathways.

IL4 and IL13 have both been regarded as attractive targets for regulating immune system. It has been proved that antagonists of IL4 and IL13 have therapeutic effects against various disorders such autoimmune diseases. However, novel inhibitors targeting IL4, IL13 or both is in demand.

### SUMMARY OF THE INVENTION

There exists a need of therapy for autoimmune disease by inhibiting IL4, IL13 or both. The present invention addresses this need and provides related advantages as well.

In one aspect, provided is a fusion polypeptide comprising a structure of formula I arranged from amino terminus to carboxyl terminus as:

X1-(S1)-X2-(S2)-X3

wherein each of S1 and S2 is independently a spacer, and each of X1, X2 and X3 is independently selected from IL13Rα2, IL4Rα and a regulatory component, with the proviso that the IL13Rα2 is closer to the amino terminus than the IL4Rα.

In some embodiments, the IL13Rα2 comprises an amino acid sequence having at least 80% identity with SEQ ID NO: 1. In some embodiments, the IL13Rα2 comprises an amino acid sequence having at least 90% identity with SEQ ID NO: 1. In some embodiments, the IL13Rα2 comprises an amino acid sequence having at least 95% identity with SEQ ID NO: 1. In some embodiments, the IL13Rα2 comprises an amino acid sequence of SEQ ID NO: 1. In some embodiments, the IL13Rα2 comprises a mutation, deletion, addition or substitution as compared to SEQ ID NO: 1. In some embodiments, the IL13Rα2 comprises a non-natural amino acid as compared to SEQ ID NO: 1. In some embodiments, the non-natural amino acid is selected from the group consisting of hydroxyproline, hydroxylysine, selenocysteine, D-type amino acids, synthetic unnatural amino acids, and derivative thereof. In some embodiments, the IL13Rα2 comprises a modification as compared to SEQ ID NO: 1. In some embodiments, the modification is present at N-terminal, C-terminal, or any amino acid residue of the IL13Rα2. In some embodiments, the modification is selected from the group consisting of pegylation, amidation, glycosylation, acylation, sulfation, phosphorylation, acetylation, cyclization, and any combination thereof.

In some embodiments, the IL4Rα comprises an amino acid sequence having at least 80% identity with SEQ ID NO: 2. In some embodiments, the IL4Rα comprises an amino acid sequence having at least 90% identity with SEQ ID NO: 2. In some embodiments, the IL4Rα comprises an amino acid sequence having at least 95% identity with SEQ ID NO: 2. In some embodiments, the IL4Rα comprises an amino acid sequence of SEQ ID NO: 2. In some embodiments, the IL4Rα comprises a mutation, deletion, addition or substitution as compared to SEQ ID NO: 2. In some embodiments, the IL4Rα comprises a non-natural amino acid as compared to SEQ ID NO: 2. In some embodiments, the non-natural amino acid is selected from the group consisting of hydroxyproline, hydroxylysine, selenocysteine, D-type amino acids, synthetic unnatural amino acids, and derivative thereof. In some embodiments, the IL4Rα comprises a modification as compared to SEQ ID NO: 2. In some embodiments, the modification is present at N-terminal, C-terminal, or any amino acid residue of the IL4Rα. In some embodiments, the modification is selected from the group consisting of pegylation, amidation, glycosylation, acylation, sulfation, phosphorylation, acetylation, cyclization, and any combination thereof.

In some embodiments, the regulatory component is selected from a group consisting of Fc domain, serum albumin, CTP, ELP, XTEN, and any fragment thereof. In some embodiments, the Fc domain is derived from IgG1, IgG2, IgG3, and IgG4. In some embodiments, the Fc domain is derived from human IgG1, IgG2, IgG3, and IgG4. In some embodiments, the Fc domain comprises an amino acid of SEQ ID NO: 6, SEQ ID NO: 7, SEQ ID NO: 8, or SEQ ID NO: 9. In some embodiments, the Fc domain further comprises a mutation, deletion, addition or substitution as compared to SEQ ID NO: 6, SEQ ID NO: 7, SEQ ID NO: 8, or SEQ ID NO: 9. In some embodiments, the serum albumin is human serum albumin (HSA). In some embodiments, the HSA comprises an amino acid of SEQ ID NO: 10. In some embodiments, the HSA further comprises a mutation, deletion, addition or substitution as compared to SEQ ID NO: 10.

In some embodiments, the fusion polypeptide functions as an antagonist of IL4, IL13, or both. In some embodiments, the regulatory component improves a pharmacokinetic property of the fusion polypeptide. In some embodiments, the regulatory component prolongs half-life of the fusion polypeptide. In some embodiments, the regulatory component prolongs in vivo half-life of the fusion polypeptide. In some embodiments, the regulatory component enhances stability of the fusion polypeptide. In some embodiments, the regulatory component enhances in vivo stability of the fusion polypeptide.

In some embodiments, the spacer is a cleavable spacer. In some embodiments, the spacer is a non-cleavable spacer. In some embodiments, the spacer is selected from a group consisting of (GS)ₙ (SEQ ID NO: 12), (GGS)ₙ (SEQ ID NO: 13), (GGGS)ₙ (SEQ ID NO: 14), (GGSG)ₙ (SEQ ID NO: 15), (GGSGG)ₙ (SEQ ID NO: 16), (GGGGS)ₙ (SEQ ID NO: 17) and null, wherein n is 1, 2, 3, 4, 5, 6, 7, 8, 9, or 10. In some embodiments, the spacer is (GGGGS)ₙ, and wherein n is 2 (SEQ ID NO: 18).

In another aspect, provided is a pharmaceutical composition comprising the fusion polypeptide as described above, and a pharmaceutically acceptable excipient. In some embodiments, the pharmaceutically acceptable excipient comprises buffer, stabilizer, preservative, tonicity agent, antioxidant, emulsifier, and viscosity-enhancing agent.

In another aspect, provided is an isolated polynucleotide encoding the fusion polypeptide as described above.

In another aspect, provided is a host cell expressing the fusion polypeptide as described above.

In another aspect, provided is a kit comprising the fusion polypeptide as described above and an instruction for using the kit.

In another aspect, provided is a method for treating an autoimmune disease comprising administrating a therapeutically effective amount of the fusion polypeptide as described above. In some embodiments, the autoimmune disease is selected from psoriasis, rheumatoid arthritis, asthma, multiple sclerosis, type-1 diabetes, inflammatory bowel diseases, Crohn's disease, Hashimoto's thyreoiditis, autoimmune thyreoiditis, autoimmune myasthenia gravis, systemic lupus erythematosus, ulcerative colitis, atopic dermatitis, myocarditis and transplantation-related diseases such as graft-versus-host or host-versus graft reactions, or general organ tolerance issues. In some embodiments, the autoimmune disease is selected from asthma and atopic dermatitis.

In another aspect, provided is use of the fusion polypeptide as described above in preparing a medicament for treating an autoimmune disease in a subject in need thereof. In some embodiments, the autoimmune disease is selected from psoriasis, rheumatoid arthritis, asthma, multiple sclerosis, type-1 diabetes, inflammatory bowel diseases, Crohn's disease, Hashimoto's thyreoiditis, autoimmune thyreoiditis, autoimmune myasthenia gravis, systemic lupus erythematosus, ulcerative colitis, atopic dermatitis, myocarditis and transplantation-related diseases such as graft-versus-host or host-versus graft reactions, or general organ tolerance issues. In some embodiments, the autoimmune disease is selected from asthma and atopic dermatitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates exemplary structural configurations of the fusion protein of the present application.

FIGS. 2A-2C and FIGS. 3A-3B illustrate the expression and purification of the fusion protein of the present application.

FIG. 4A illustrates the IL13Rα2/IL4Rα fusion protein of the present application blocks stimulation of IL4 on TF-1 cell proliferation. FIG. 4B illustrates the IL13Rα2/IL4Rα fusion protein of the present application blocks stimulation of IL13 on TF-1 cell proliferation.

FIG. 5A and FIG. 5B illustrate mutual interferences of IL13 and IL4 on each other's binding with the fusion protein of the present application.

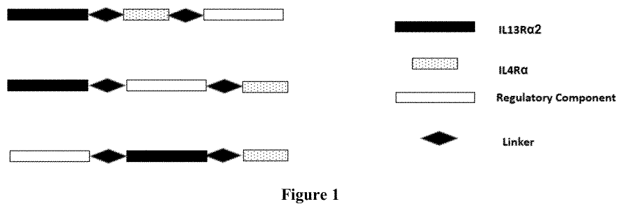

### DETAILED DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

As used in the specification and claims, the singular forms “a”, “an” and “the” include plural references unless the context clearly dictates otherwise.

### Definition

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “amino acid” refers to either natural and/or unnatural or synthetic amino acids, including but not limited to both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

A “fragment” when applied to a protein, is a truncated form of a native biologically active protein that may or may not retain at least a portion of the therapeutic and/or biological activity. A “variant” when applied to a protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein. As used herein, the term “biologically active protein moiety” includes proteins modified deliberately, as for example, by site directed mutagenesis, synthesis of the encoding gene, insertions, or accidentally through mutations.

“Conjugated”, “linked,” “fused,” and “fusion” are used interchangeably herein and refer to the joining together of two or more chemical elements, sequences or components, by whatever means including chemical conjugation or recombinant means. For example, a promoter or enhancer is operably linked to a coding sequence if it affects the transcription of the sequence. Generally, “operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. Thus, the resulting “fusion polypeptide” is a single protein containing two or more fragments that correspond to polypeptides encoded by the original ORFs (which segments are not normally so joined in nature). The “fusion site” refers to the sequence where the two or more fragments are joined together. In some cases, the fusion site can be a sequence that is identical in the two or more fragments. For example, the fusion site can be a sequence of about 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids that is identical in the joined fragments. In specific examples, the fusion site can be a sequence of about 6, 7, 8, 9, 10, 11, 12, 13, 14, 15, 16, 17, 18, 19, 20, 21, 22, 23 or 24 amino acids that is identical in the joined fragments.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

“Homology” or “homologous” or “sequence identity” refers to sequence similarity or interchangeability between two or more polynucleotide sequences or between two or more polypeptide sequences. When using a program such as Emboss Needle or BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity compared to those sequences. Polypeptides that are homologous preferably have sequence identities of at least 80%, or at least 90%, or at least 95%, or at least 97%, or at least 98%, or have at least 99% sequence identity when sequences of comparable length are optimally aligned.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, NEEDLE or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “antagonist” and “inhibitor” are used interchangeably herein and refer to a molecule capable of inhibiting a biological function of a target protein, whether by inhibiting the activity, or expression of the target protein. Accordingly, the terms “antagonist” and “inhibitors” are defined in the context of the biological role of the target protein. While preferred antagonists herein specifically interact with (e.g. bind to) the target, molecules that inhibit a biological activity of the target protein by interacting with other members of the signaling pathway of which the target protein is a member are also specifically included within this definition.

The term “effective amount” or “therapeutically effective amount” refers to an amount of the fusion polypeptide described herein that is sufficient to effect the intended application including but not limited to disease treatment. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. inhibition of cell proliferation. The specific dose will vary depending on the particular fusion polypeptide chosen, the dosing regimen to be followed, whether it is administered in combination with other drugs, timing of administration, the tissue to which it is administered, and the physical delivery system in which it is carried.

The term “treatment” or “treating,” or “palliating” or “ameliorating” can be used interchangeably herein and refer to an approach for obtaining beneficial or desired results including but not limited to a therapeutic benefit and/or a prophylactic benefit. By therapeutic benefit, it means eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal so that both agents and/or their metabolites are present in the subject at the same time. Co-administration includes simultaneous administration in separate compositions, administration at different times in separate compositions, or administration in a composition in which both agents are present.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like, when the molecule contains an acidic functionality; and when the molecule contains a basic functionality, salts of organic or inorganic acids, such as hydrochloride, hydrobromide, tartrate, mesylate (methane sulfonate), ethane sulfonate, acetate, maleate, oxalate, phosphate, and the like. In a compound with more than one basic moiety, more than one of the basic moieties may be converted to the salt form, including but not limited to a bis- or tris-salt. Alternatively, a compound having more than one basic moiety may form a salt at only one of the basic moieties.

The term “in vivo” refers to an event that takes place in a subject's body.

The term “in vitro” refers to an event that takes places outside of a subject's body. For example, an in vitro assay encompasses any assay run outside of a subject assay. in vitro assays encompass cell-based assays in which cells alive or dead are employed. in vitro assays also encompass a cell-free assay in which no intact cells are employed.

### Fusion Polypeptides

In one aspect, the present disclosure relates to fusion polypeptides as an antagonist of IL4, IL13 or both.

Interleukin 4 (IL-4, IL4) is a cytokine expressed on mast cells, basophils, a subset of activated T cells, eosinophils and neutrophils. IL4 has been shown to play many biological roles, including the stimulation of activated B-cell and T-cell proliferation, and the differentiation of B cells into plasma cells. IL4 is a key regulator in humoral and adaptive immunity. It can induce B-cell class switching to IgE, and up-regulate MHC class II production. IL4 also decreases the production of Th1 cells, macrophages, IFN-gamma, and IL-12. IL4 is involved in the development of many immune disorders, particularly allergies and some autoimmune diseases. Human IL4 is a 129 amino acid glycoprotein. The amino acid sequence of human IL4 is as shown in SEQ ID NO: 4. The biological activities of IL4 are effected through binding to its cell surface receptors.

The receptor of IL4 is known as IL4Rα. This receptor exists in 2 different complexes throughout the body. Type I receptor is composed of an IL4Rα subunit (SEQ ID NO: 2) and a common γ chain. Type II receptor is composed of an IL4Rα subunit and an IL13 receptor known as IL13Rα1 (SEQ ID NO: 3). This type II receptor has the ability to bind both IL4 and IL13, two cytokines with closely related biological functions.

Interleukin 13 (IL-13, IL13) is a cytokine secreted by T helper type 2 (Th2) cells, CD4 cells, natural killer T cell, mast cells, basophils, eosinophils and nuocytes. IL13 is a central regulator in IgE synthesis, goblet cell hyperplasia, mucus hypersecretion, airway hyperresponsiveness, fibrosis and chitinase up-regulation. IL13 is involved in different diseases including inflammation and asthma. Human IL13 has 111 amino acids, and the amino acid sequence of human IL13 is as shown in SEQ ID NO: 5.

The signaling of IL13 begins through a shared receptor with IL4. This receptor is a heterodimer receptor complex consisting of IL4Rα (SEQ ID NO: 2) and IL13Rα1 (SEQ ID NO: 3). The binding of IL13 to the IL13Rα1 further increases the probability of a heterodimer formation to IL4Rα and the production of the type 2 IL4 receptor, and eventually allows for the downstream activation of JAK-STAT6 signaling pathway.

IL13 also binds to another receptor known as IL13Rα2 (SEQ ID NO: 1). IL13Rα2 is derived from Th2 cells, which is considered as a decoy receptor. The IL13Rα2 subunit binds only to IL13, and exists in both membrane-bound and soluble forms in mice. However, soluble form of IL13Rα2 has not been detected in human.

In one aspect, provided herein is a fusion polypeptide comprising a structure of formula I arranged from amino terminus to carboxyl terminus as:

### X1-(S1)-X2-(S2)-X3

wherein each of S1 and S2 is independently a spacer, and each of X1, X2 and X3 is independently selected from IL13Rα2, IL4Rα and a regulatory component (RC), with the proviso that the IL13Rα2 is closer to the amino terminus than the IL4Rα.

In some embodiments, provided herein is a fusion polypeptide comprising a structure of IL13Rα2-(S1)-IL4Rα-(S2)-RC, wherein each of S1 and S2 is independently a spacer. In some embodiments, provided herein is a fusion polypeptide comprising a structure of RC-(S1)-IL13Rα2-(S2)-IL4Rα, wherein each of S1 and S2 is independently a spacer. In some embodiments, provided herein is a fusion polypeptide comprising a structure of IL13Rα2-(S1)-RC-(S2)-IL4Rα, wherein each of S1 and S2 is independently a spacer.

S1 and S2 of the fusion polypeptide each can be any suitable spacers used for connecting the structural components of the fusion polypeptide herein. In some embodiments, the spacer is a cleavable spacer. In some embodiments, the spacer is a non-cleavable spacer. In some embodiments, S1 and S2 of the fusion polypeptide have the same structure. In some embodiments, S1 and S2 of the fusion polypeptide have different structures.

In some embodiments, the spacer is selected from a group consisting of (GS)ₙ (SEQ ID NO: 12), (GGS)ₙ (SEQ ID NO: 13), (GGGS)ₙ (SEQ ID NO: 14), (GGSG)ₙ (SEQ ID NO: 15), (GGSGG)ₙ (SEQ ID NO: 16), (GG-GGS)ₙ. (SEQ ID NO: 17) and null, wherein n is 1, 2, 3, 4, 5, 6, 7, 8, 9, or 10. In some embodiments, the spacer is (GS)ₙ, wherein n is 1, 2, 3, 4, 5, 6, 7, 8, 9, or 10 (SEQ ID NO: 12). In some embodiments, the spacer is (GS)ₙ, wherein n is 2 (SEQ ID NO: 19). In some embodiments, the spacer is GSGS (SEQ ID NO: 19). In some embodiments, the spacer is (GGS)ₙ, wherein n is 1, 2, 3, 4, 5, 6, 7, 8, 9, or 10 (SEQ ID NO: 13). In some embodiments, the spacer is (GGS)ₙ, wherein n is 2 (SEQ ID NO: 20). In some embodiments, the spacer is GGSGGS (SEQ ID NO: 20). In some embodiments, the spacer is (GGGS)ₙ, wherein n is 1, 2, 3, 4, 5, 6, 7, 8, 9, or 10 (SEQ ID NO: 14). In some embodiments, the spacer is (GGGS)ₙ, wherein n is 2 (SEQ ID NO: 21). In some embodiments, the spacer is GGGSGGGS (SEQ ID NO: 21). In some embodiments, the spacer is (GGSG)ₙ, wherein n is 1, 2, 3, 4, 5, 6, 7, 8, 9, or 10 (SEQ. ID NO: 15). In some embodiments, the spacer is (GGSG)ₙ, wherein n is 2 (SEQ ID NO: 23). In some embodiments, the spacer is GGSGGGSG (SEQ ID NO: 23). In some embodiments, the spacer is (GGSGG)ₙ, wherein n is 1, 2, 3, 4, 5, 6, 7, 8, 9, or 10 (SEQ ID NO: 16). In some embodiments, the spacer is (GGSGG)ₙ, wherein n is 2 (SEQ ID NO: 22). In some embodiments, the spacer is GGSGGGGSGG (SEQ ID NO: 22). In some embodiments, the spacer is (GGGGS)ₙ, wherein n is 1, 2, 3, 4, 5, 6, 7, 8, 9, or 10 (SEQ ID NO: 17). In some embodiments, the spacer is (GGGGS)ₙ, wherein n is 2 (SEQ ID NO: 18). In some embodiments, the spacer is GGGGSGGGGS (SEQ ID NO: 18).

In some embodiments, S1 is GGGGSGGGGS (SEQ ID NO: 18). In some embodiments, S2 is GGGGSGGGGS (SEQ ID NO: 18). In some embodiments, S1 is GGGGSGGGGS (SEQ 11) NO: 18), S2 is GGGGSGGGGS (SEQ ID NO: 18), and the fusion polypeptide comprises a structure of IL13Rα2-GGGSGGGS-IL4Rα-GGGSGGGS-RC. In some embodiments, S1 is GGGGSGGGGS (SEQ ID NO: 18), S2 is GGGGSGGGGS (SEQ ID NO: 18), and the fusion polypeptide comprises a structure of RC-GGGSGGGS-IL13Rα2-GGGSGGGS-IL4Rα. In some embodiments, S1 is GGGGCSCGGGGS (SEQ ID NO: 18), S2 is GGGGSGGGGS (SEQ ID NO: 18), and the fusion polypeptide comprises a structure of IL13Rα2-GGGSGGGS-RC-GGGSGGGS-IL4Rα.

In some embodiments, the IL13Rα2 used in the fusion polypeptide herein is derived from human. In some embodiments, the IL13Rα2 of the fusion polypeptide comprises an amino acid sequence having at least 70% identity with SEQ ID NO: 1. In some embodiments, the IL13Rα2 of the fusion polypeptide comprises an amino acid sequence having at least 75% identity with SEQ ID NO: 1. In some embodiments, the IL13Rα2 of the fusion polypeptide comprises an amino acid sequence having at least 70% identity with SEQ ID NO: 1. In some embodiments, the IL13Rα2 of the fusion polypeptide comprises an amino acid sequence having at least 80% identity with SEQ ID NO: 1. In some embodiments, the IL13Rα2 of the fusion polypeptide comprises an amino acid sequence having at least 85% identity with SEQ ID NO: 1. In some embodiments, the IL13Rα2 of the fusion polypeptide comprises an amino acid sequence having at least 90% identity with SEQ ID NO: 1. In some embodiments, the IL13Rα2 of the fusion polypeptide comprises an amino acid sequence having at least 91% identity with SEQ ID NO: 1. In some embodiments, the IL13Rα2 of the fusion polypeptide comprises an amino acid sequence having at least 92% identity with SEQ ID NO: 1. In some embodiments, the IL13Rα2 of the fusion polypeptide comprises an amino acid sequence having at least 93% identity with SEQ ID NO: 1. In some embodiments, the IL13Rα2 of the fusion polypeptide comprises an amino acid sequence having at least 94% identity with SEQ ID NO: 1. In some embodiments, the IL13Rα2 of the fusion polypeptide comprises an amino acid sequence having at least 95% identity with SEQ ID NO: 1. In some embodiments, the IL13Rα2 of the fusion polypeptide comprises an amino acid sequence having at least 96% identity with SEQ ID NO: 1. In some embodiments, the IL13Rα2 of the fusion polypeptide comprises an amino acid sequence having at least 97% identity with SEQ ID NO: 1. In some embodiments, the IL13Rα2 of the fusion polypeptide comprises an amino acid sequence having at least 98% identity with SEQ ID NO: 1. In some embodiments, the IL13Rα2 of the fusion polypeptide comprises an amino acid sequence having at least 99% identity with SEQ ID NO: 1. In some embodiments, the IL13Rα2 of the fusion polypeptide comprises an amino acid sequence of SEQ ID NO: 1.

In some embodiments, the IL13Rα2 of the fusion polypeptide comprises a mutation, deletion, addition or substitution as compared to SEQ ID NO: 1. In some embodiments, the IL13Rα2 of the fusion polypeptide comprises one or more mutations as compared to SEQ ID NO: 1. In some embodiments, introduction of the mutation can improve the activity of the IL13Rα2 component of the fusion polypeptide. In some embodiments, introduction of the mutation can enhance the affinity of the IL13Rα2 component for IL13. In some embodiments, introduction of the mutation can improve the pharmacokinetic property of the fusion polypeptide. In some embodiments, introduction of the mutation can prolong half-life of the fusion polypeptide and/or enhances stability of the fusion polypeptide.

In some embodiments, the IL13Rα2 of the fusion polypeptide comprises one or more deletions as compared to SEQ ID NO: 1. In some embodiments, introduction of the deletion can improve the activity of the IL13Rα2 component of the fusion polypeptide. In some embodiments, introduction of the deletion can enhance the affinity of the IL13Rα2 component for IL13. In some embodiments, introduction of the deletion can improve the pharmacokinetic property of the fusion polypeptide. In some embodiments, introduction of the deletion can prolong half-life of the fusion polypeptide and/or enhances stability of the fusion polypeptide.

In some embodiments, the IL13Rα2 of the fusion polypeptide comprises one or more additions as compared to SEQ ID NO: 1. In some embodiments, introduction of the addition can improve the activity of the IL13Rα2 component of the fusion polypeptide. In some embodiments, introduction of the addition can enhance the affinity of the IL13Rα2 component for IL13. In some embodiments, introduction of the addition can improve the pharmacokinetic property of the fusion polypeptide. In some embodiments, introduction of the addition can prolong half-life of the fusion polypeptide and/or enhances stability of the fusion polypeptide.

In some embodiments, the IL13Rα2 of the fusion polypeptide comprises one or more substitutions as compared to SEQ ID NO: 1. In some embodiments, introduction of the substitution can improve the activity of the IL13Rα2 component of the fusion polypeptide. In some embodiments, introduction of the substitution can enhance the affinity of the IL13Rα2 component for IL13. In some embodiments, introduction of the substitution can improve the pharmacokinetic property of the fusion polypeptide. In some embodiments, introduction of the substitution can prolong half-life of the fusion polypeptide and/or enhances stability of the fusion polypeptide.

In some embodiments, the IL13Rα2 of the fusion polypeptide comprises one or more non-natural amino acid as compared to SEQ ID NO: 1. In some embodiments, said non-natural amino acid is selected from the group consisting of hydroxyproline, hydroxylysine, selenocysteine, D-type amino acids, synthetic unnatural amino acids, and derivative thereof. In some embodiments, introduction of the non-natural amino acid can improve the activity of the IL13Rα2 component of the fusion polypeptide. In some embodiments, introduction of the non-natural amino acid can enhance the affinity of the IL13Rα2 component for IL13. In some embodiments, introduction of the non-natural amino acid can improve the pharmacokinetic property of the fusion polypeptide. In some embodiments, introduction of the non-natural amino acid can prolong half-life of the fusion polypeptide and/or enhances stability of the fusion polypeptide.

In some embodiments, the IL13Rα2 of the fusion polypeptide comprises a modification as compared to SEQ ID NO: 1. In some embodiments, said is selected from the group consisting of pegylation, amidation, glycosylation, acylation, sulfation, phosphorylation, acetylation, cyclization, and any combination thereof. The modification can be present at N-terminal, C-terminal, or any amino acid residue of the IL13Rα2. In some embodiments, the modification is pegylation. Said pegylation can be present at N-terminal, C-terminal, or any amino acid residue of the IL13Rα2. In some embodiments, introduction of the modification can improve the pharmacokinetic property of the fusion polypeptide. In some embodiments, introduction of the modification can prolong half-life of the fusion polypeptide and/or enhances stability of the fusion polypeptide.

In some embodiments, the IL4Rα used in the fusion polypeptide herein is derived from human. In some embodiments, the IL4Rα of the fusion polypeptide comprises an amino acid sequence having at least 70% identity with SEQ ID NO: 2. In some embodiments, the IL4Rα of the fusion polypeptide comprises an amino acid sequence having at least 75% identity with SEQ ID NO: 2. In some embodiments, the IL4Rα of the fusion polypeptide comprises an amino acid sequence having at least 80% identity with SEQ ID NO: 2. In some embodiments, the IL4Rα of the fusion polypeptide comprises an amino acid sequence having at least 85% identity with SEQ ID NO: 2. In some embodiments, the IL4Rα of the fusion polypeptide comprises an amino acid sequence having at least 90% identity with SEQ ID NO: 2. In some embodiments, the IL4Rα of the fusion polypeptide comprises an amino acid sequence having at least 91% identity with SEQ ID NO: 2. In some embodiments, the IL4Rα of the fusion polypeptide comprises an amino acid sequence having at least 92% identity with SEQ ID NO: 2. In some embodiments, the IL4Rα of the fusion polypeptide comprises an amino acid sequence having at least 93% identity with SEQ ID NO: 2. In some embodiments, the IL4Rα of the fusion polypeptide comprises an amino acid sequence having at least 94% identity with SEQ ID NO: 2. In some embodiments, the IL4Rα of the fusion polypeptide comprises an amino acid sequence having at least 95% identity with SEQ ID NO: 2. In some embodiments, the IL4Rα of the fusion polypeptide comprises an amino acid sequence having at least 96% identity with SEQ ID NO: 2. In some embodiments, the IL4Rα of the fusion polypeptide comprises an amino acid sequence having at least 97% identity with SEQ ID NO: 2. In some embodiments, the IL4Rα of the fusion polypeptide comprises an amino acid sequence having at least 98% identity with SEQ ID NO: 2. In some embodiments, the IL4Rα of the fusion polypeptide comprises an amino acid sequence having at least 99% identity with SEQ ID NO: 2. In some embodiments, the IL4Rα of the fusion polypeptide comprises an amino acid sequence of SEQ ID NO: 2.

In some embodiments, the IL4Rα of the fusion polypeptide comprises a mutation, deletion, addition or substitution as compared to SEQ ID NO: 2. In some embodiments, the IL4Rα of the fusion polypeptide comprises one or more mutations as compared to SEQ ID NO: 2. In some embodiments, introduction of the mutation can improve the activity of the IL4Rα component of the fusion polypeptide. In some embodiments, introduction of the mutation can enhance the affinity of the IL4Rα component for IL4. In some embodiments, introduction of the mutation can improve the pharmacokinetic property of the fusion polypeptide. In some embodiments, introduction of the mutation can prolong half-life of the fusion polypeptide and/or enhances stability of the fusion polypeptide.

In some embodiments, the IL4Rα of the fusion polypeptide comprises one or more deletions as compared to SEQ ID NO: 2. In some embodiments, introduction of the deletion can improve the activity of the IL4Rα component of the fusion polypeptide. In some embodiments, introduction of the deletion can enhance the affinity of the IL4Rα component for IL4. In some embodiments, introduction of the deletion can improve the pharmacokinetic property of the fusion polypeptide. In some embodiments, introduction of the deletion can prolong half-life of the fusion polypeptide and/or enhances stability of the fusion polypeptide.

In some embodiments, the IL4Rα of the fusion polypeptide comprises one or more additions as compared to SEQ ID NO: 2. In some embodiments, introduction of the addition can improve the activity of the IL4Rα component of the fusion polypeptide. In some embodiments, introduction of the addition can enhance the affinity of the IL4Rα component for IL4. In some embodiments, introduction of the addition can improve the pharmacokinetic property of the fusion polypeptide. In some embodiments, introduction of the addition can prolong half-life of the fusion polypeptide and/or enhances stability of the fusion polypeptide.

In some embodiments, the IL4Rα of the fusion polypeptide comprises one or more substitutions as compared to SEQ ID NO: 2. In some embodiments, introduction of the substitution can improve the activity of the IL4Rα component of the fusion polypeptide. In some embodiments, introduction of the substitution can enhance the affinity of the IL4Rα component for IL4. In some embodiments, introduction of the substitution can improve the pharmacokinetic property of the fusion polypeptide. In some embodiments, introduction of the substitution can prolong half-life of the fusion polypeptide and/or enhances stability of the fusion polypeptide.

In some embodiments, the IL4Rα of the fusion polypeptide comprises one or more non-natural amino acid as compared to SEQ ID NO: 2. In some embodiments, said non-natural amino acid is selected from the group consisting of hydroxyproline, hydroxylysine, selenocysteine, D-type amino acids, synthetic unnatural amino acids, and derivative thereof. In some embodiments, introduction of the non-natural amino acid can improve the activity of the IL4Rα component of the fusion polypeptide. In some embodiments, introduction of the non-natural amino acid can enhance the affinity of the IL4Rα component for IL4. In some embodiments, introduction of the non-natural amino acid can improve the pharmacokinetic property of the fusion polypeptide. In some embodiments, introduction of the non-natural amino acid can prolong half-life of the fusion polypeptide and/or enhances stability of the fusion polypeptide.

In some embodiments, the IL4Rα of the fusion polypeptide comprises a modification as compared to SEQ ID NO: 2. In some embodiments, said is selected from the group consisting of pegylation, amidation, glycosylation, acylation, sulfation, phosphorylation, acetylation, cyclization, and any combination thereof. The modification can be present at N-terminal, C-terminal, or any amino acid residue of the IL4Rα. In some embodiments, the modification is pegylation. Said pegylation can be present at N-terminal, C-terminal, or any amino acid residue of the IL4Rα. In some embodiments, introduction of the modification can improve the pharmacokinetic property of the fusion polypeptide. In some embodiments, introduction of the modification can prolong half-life of the fusion polypeptide and/or enhances stability of the fusion polypeptide.)

The regulatory component (RC) of the fusion polypeptide herein can be any structural moiety capable of improving one or more pharmacokinetic properties of the fusion polypeptide. In some embodiments, the regulatory component can be selected from the group consisting of Fc domain, serum albumin, CTP, ELP, XTEN, and any fragment thereof.

In some embodiments, the regulatory component is an Fc domain. In some embodiments, said Fc domain is derived from IgG1, IgG3, and IgG4. In some embodiments, the Fc domain is derived from the Fc region of IgG1. In some embodiments, the Fc domain comprises an amino acid sequence having at least 70%, 80%, 90%, 95%, 96%, 97%, 98%, or 99% identity with the Fc region of human IgG1. In some embodiments, the Fc domain comprises an amino acid sequence of the Fc region of human IgG1. In some embodiments, the Fc domain comprises an amino acid sequence having one or more mutations, deletions, additions, substitutions or any combination thereof as compared with the Fc region of human IgG1. In some embodiments, the Fc domain comprises an amino acid sequence having at least 70%, 80%, 90%, 95%, 96%, 97%, 98%, or 99% identity with SEQ ID NO: 6. In some embodiments, the Fc domain comprises an amino acid sequence of SEQ ID NO: 6. In some embodiments, the Fc domain comprises an amino acid sequence having one or more mutations, deletions, additions, substitutions or any combination thereof as compared with SEQ ID NO: 6. In some embodiments, the Fc domain comprises an amino acid sequence having at least 70%, 80%, 90%, 95%, 96%, 97%, 98%, or 99% identity with SEQ ID NO: 7. In some embodiments, the Fc domain comprises an amino acid sequence of SEQ ID NO: 7. In some embodiments, the Fc domain comprises an amino acid sequence having one or more mutations, deletions, additions, substitutions or any combination thereof as compared with SEQ ID NO: 7.

In some embodiments, the Fc domain is derived from the Fc region of IgG2. In some embodiments, the Fc domain comprises an amino acid sequence having at least 70%, 80%, 90%, 95%, 96%, 97%, 98%, or 99% identity with the Fc region of human IgG2. In some embodiments, the Fc domain comprises an amino acid sequence of the Fc region of human IgG2. In some embodiments, the Fc domain comprises an amino acid sequence having one or more mutations, deletions, additions, substitutions or any combination thereof as compared with the Fc region of human IgG2. In some embodiments, the Fc domain comprises an amino acid sequence having at least 70%, 80%, 90%, 95%, 96%, 97%, 98%, or 99% identity with SEQ ID NO: 8. In some embodiments, the Fc domain comprises an amino acid sequence of SEQ ID NO: 8. In some embodiments, the Fc domain comprises an amino acid sequence having one or more mutations, deletions, additions, substitutions or any combination thereof as compared with SEQ ID NO: 8.

In some embodiments, the Fc domain is derived from the Fc region of IgG3. In some embodiments, the Fc domain comprises an amino acid sequence having at least 70%, 80%, 90%, 95%, 96%, 97%, 98%, or 99% identity with the Fc region of human IgG3. In some embodiments, the Fc domain comprises an amino acid sequence of the Fc region of human IgG3. In some embodiments, the Fc domain comprises an amino acid sequence having one or more mutations, deletions, additions, substitutions or any combination thereof as compared with the Fc region of human IgG3.

In some embodiments, the Fc domain is derived from the Fc region of IgG4. In some embodiments, the Fc domain comprises an amino acid sequence having at least 70%, 80%, 90%, 95%, 96%, 97%, 98%, or 99% identity with the Fc region of human IgG4. In some embodiments, the Fc domain comprises an amino acid sequence of the Fc region of human IgG4. In some embodiments, the Fc domain comprises an amino acid sequence having one or more mutations, deletions, additions, substitutions or any combination thereof as compared with the Fc region of human IgG4. In some embodiments, the Fc domain comprises an amino acid sequence having at least 70%, 80%, 90%, 95%, 96%, 97%, 98%, or 99% identity with SEQ ID NO: 9 (human IgG4 S228P). In some embodiments, the Fc domain comprises an amino acid sequence of SEQ ID NO: 9. In some embodiments, the Fc domain comprises an amino acid sequence having one or more mutations, deletions, additions, substitutions or any combination thereof as compared with SEQ ID NO: 9

In some embodiments, said regulatory component is derived from serum albumin or a fragment thereof. In some embodiments, said regulatory component is derived from human serum albumin (HSA) or a fragment thereof. In some embodiments, the regulatory component comprises an amino acid sequence having one or more mutations, deletions, additions, substitutions or any combination thereof as compared with the HSA. In some embodiments, the regulatory component comprises an amino acid sequence having at least 70%, 80%, 90%, 95%, 96%, 97%, 98%, or 99% identity with SEQ ID NO: 10. In some embodiments, the regulatory component comprises an amino acid sequence of SEQ ID NO: 10. In some embodiments, the regulatory component comprises an amino acid sequence having one or more mutations, deletions, additions, substitutions or any combination thereof as compared with SEQ ID NO: 10.

In some embodiments, the regulatory component prolongs half-life of the fusion polypeptide. In some embodiments, the regulatory component prolongs in vivo half-life of the fusion polypeptide. In some embodiments, the regulatory component prolongs in vivo half-life of the fusion polypeptide by at least 0.5 hour, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 day, 3 day, 4 day, 5 day, 6 day, 1 week, 2 week, or 1 months.

In some embodiments, the regulatory component enhances stability of the fusion polypeptide. In some embodiments, the regulatory component enhances in vivo stability of the fusion polypeptide. In some embodiments, the regulatory component enhances in vitro stability of the fusion polypeptide. In some embodiments, the regulatory component increases the solubility of the fusion polypeptide. In some embodiments, the regulatory component increases the in vivo solubility of the fusion polypeptide. In some embodiments, the regulatory component increases the in vitro solubility of the fusion polypeptide. In some embodiments, the regulatory component increases the heat solubility of the fusion polypeptide. In some embodiments, the regulatory component increases the in vivo heat solubility of the fusion polypeptide. In some embodiments, the regulatory component increases the in vitro heat solubility of the fusion polypeptide. In some embodiments, the regulatory component reduces the aggregation of the fusion polypeptide. In some embodiments, the regulatory component reduces the in vivo aggregation of the fusion polypeptide. In some embodiments, the regulatory component reduces the in vitro aggregation of the fusion polypeptide.

In some embodiments, the regulatory component improves the bioavailability of the fusion polypeptide. In some embodiments, the regulatory component improves the bioavailability of the fusion polypeptide by at least 1%, 2%, 3%, 4%, 5%, 6%, 7%, 8%, 9%, 10%, 15%, 20%, 25%, 30%, 35%, 40%, 45%, 50%, 60%, 70%.

In some embodiments, the regulatory component improves the activity of one or more other structural components of the fusion polypeptide. In some the regulatory component improves the activity of the IL13Rα2 component of the fusion polypeptide. In some the regulatory component improves the activity of the IL4Rα component of the fusion polypeptide. In some the regulatory component improves the activity of the IL13Rα2 and the IL4 Rα component of the fusion polypeptide. In some embodiments, the regulatory component improves the specificity of one or more other structural components of the fusion polypeptide. In some the regulatory component improves the specificity of the IL13Rα2 component for IL13. In some the regulatory component improves the specificity of the IL4Rα component for IL4. In some the regulatory component improves the specificity of the IL13Rα2 component for IL13 and the specificity of the IL4Rα component for IL4.

In some embodiments, the fusion polypeptide herein comprises a structural of IL13Rα2-(GGGGS)₂-IL4Rα-(GG-GG-S)₂-RC. In some embodiments, the fusion polypeptide herein comprises a structural of IL13Rα2-(GGGGS)₂-IL4Rα-(GGGGS)₂-hIgG4 Fc. In some embodiments, the fusion polypeptide herein comprises a structural of IL13Rα2-(GGGGS)₂-IL4Rα-(GGGGS)₂-hIgG4 Fc S228P. In some embodiments, the fusion polypeptide herein comprises an amino acid sequence having at least 70%, 80%, 90%, 95%, 96%, 97%, 98%, or 99% identity with SEQ ID NO: 11. In some embodiments, the fusion polypeptide herein comprises an amino acid sequence of SEQ ID NO: 11. In some embodiments, the fusion polypeptide herein comprises an amino acid sequence having one or more mutations, deletions, additions, substitutions or any combination thereof as compared with SEQ ID NO: 11.

In another aspect, provided is isolated polynucleotide encoding the fusion polypeptide of the present application.

The polynucleotide encoding the fusion polypeptide of the present application can be expressed by host cells. A host cell includes an individual cell, cell culture, or cell line. In some embodiments, host cells include progeny of a single host cell. A host cell can be transfected with a heterologous sequence including vectors comprising the polynucleotide encoding the fusion polypeptide of the present disclosure. Host cells may be prokaryotic or eukaryotic, such as bacterial cells, fungal cells, animal cells, insect cells, plant cells and the like. Examples of bacterial host cells include microorganisms belonging to the genus Escherichia, Serratia, Bacillus, Brevibacterium, Corynebacterium, Microbacterium, Pseudomonas and the like. For example, bacterial host cells may include, but not be limited to, Escherichia coli XL1-Blue, XL2-Blue, DH1, MC1000, KY3276, W1485, JM109, HB101, No. 49, i W3110, NY49, G1698, BL21, or TB1. Other bacterial host cells may include, but not be limited to, Serratia ficaria, Serratia fonticola, Serratia liquefaciens, Serratia marcescens, Bacillus subtilis, Bacillus amyloliquefaciens, Brevibacterium ammoniagenes, Brevibacterium immariophilum ATCC 14068, Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 13869, Corynebacterium acetoacidophilum ATCC 13870, Microbacterium ammoniaphilum ATCC 15354, Pseudomonas putida, Pseudomonas sp. D-0110 and the like.

Yeast host cells may include microorganisms belonging to the genus Kluyveromyces, Trichosporon, Saccharomyces, Schizosaccharomyces, Schwanniomyces, Pichia, Candida and the like, such as Saccharomyces cerevisiae, Schizosaccharomyces pombe, Kluyveromyces lactis, Trichosporon pullulans, Schwanniomyces alluvius, Candida utilis and the like.

Examples of eukaryotic cells include animal cells such as mammalian cells. For example, host cells include, but are not limited to, Chinese hamster ovary cells (CHO) or monkey cells, such as COS cells, HepG2 cells, A549 cells, and any cells that are available through ATCC or other depositories.

The host cells may be grown in cultures, and in any apparatus that may be used to grow cultures, including fermentors. They may be grown as monolayers or attached to a surface. Alternatively, the host cells may be grown in suspension. The cells can be grown in a culture medium that is serum-free. The media can be a commercially available media, such as, but not limited to, Opti-CHO (Invitrogen, Catalogue #12681) supplemented with glutamine, such as 8 mM L-glutamine.

The host cells may comprise a heterologous sequence to effect expression of the fusion polypeptides. The heterologous sequence may comprise a vector, which is a nucleic acid molecule, preferably self-replicating, which transfers an inserted nucleic acid molecule into and/or between host cells. Vectors may include those that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An expression vector is a polynucleotide which, when introduced into an appropriate host cell, can be transcribed and translated into a polypeptide(s).

The heterologous sequence encoding a fusion polypeptide of the present invention can be expressed by a single or multiple vectors. The nucleic acid sequences can be arranged in any order in a single operon, or in separate operons that are placed in one or multiple vectors. Where desired, two or more expression vectors can be employed, each of which contains one or more heterologous sequences operably linked in a single operon. Linked refers to the joining together of two more chemical elements or components, by whatever means including chemical conjugation or recombinant means. Operably-linked refers to a juxtaposition wherein the components so described are in a relationship permitting them to function in their intended manner. For instance, a promoter sequence is linked, or operably linked, to a coding sequence if the promoter sequence promotes transcription of the coding sequence. The subject vectors can stay replicable episomally, or as an integral part of the host cell genome.

The heterologous sequences of the present disclosure can be under the control of a single regulatory element. In some cases, the heterologous nucleic acid sequences are regulated by a single promoter. In other cases, the heterologous nucleic acid sequences are placed within a single operon. In still other cases, the heterologous nucleic acid sequences are placed within a single reading frame.

Preparation of the polynucleotide herein can be carried out by a variety of routine recombinant techniques and synthetic procedures. Standard recombinant DNA and molecular cloning techniques are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M L. Bennan, and L. W. Enquist, Experiments with Gene Fusions, Cold Spring Harbor Laboratory, Cold Spring Harbor, N.Y. (1984) and by Ausubel, F. M et al., Current Protocols in Molecular Biology, pub. by Greene Publishing Assoc. and Wiley-Interscience (1987). Briefly, the subject nucleic acids can be prepared genomic DNA fragments, cDNAs, and RNAs, all of which can be extracted directly from a cell or recombinantly produced by various amplification processes including but not limited to PCR and rt-PCR.

Direct chemical synthesis of nucleic acids typically involves sequential addition of 3′-blocked and 5′-blocked nucleotide monomers to the terminal 5′-hydroxyl group of a growing nucleotide polymer chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (for example, Matteuci et al., Tet. Lett. 521:719 (1980); U.S. Pat. No. 4,500,707 to Caruthers et al.; and U.S. Pat. Nos. 5,436,327 and 5,700,637 to Southern et al.).

Regulatory elements include, for example, promoters and operators, which can also be engineered to increase the expression of one or more heterologous sequences encoding a glycoprotein. A promoter is a sequence of nucleotides that initiates and controls the transcription of a nucleic acid sequence by an RNA polymerase enzyme. An operator is a sequence of nucleotides adjacent to the promoter that functions to control transcription of the desired nucleic acid sequence. The operator contains a protein-binding domain where a specific repressor protein can bind. In the absence of a suitable repressor protein, transcription initiates through the promoter. In the presence of a suitable repressor protein, the repressor protein binds to the operator and thereby inhibits transcription from the promoter.

In some embodiments of the present disclosure, promoters used in expression vectors are inducible. In other embodiments, the promoters used in expression vectors are constitutive. In some embodiments, one or more nucleic acid sequences are operably linked to an inducible promoter, and one or more other nucleic acid sequences are operably linked to a constitutive promoter. Non-limiting examples of suitable promoters for use in eukaryotic host cells include, but are not limited to, a CMV immediate early promoter, an HSV thymidine kinase promoter, an early or late SV40 promoter, LTRs from retroviruses, and a mouse metallothionein-I promoter.

The genes in the expression vector typically will also encode a ribosome binding site to direct translation (that is, synthesis) of any encoded mRNA gene product. Other regulatory elements that may be used in an expression vector include transcription enhancer elements and transcription terminators. See, for example, Bitter et al., Methods in Enzymology, 153:516-544 (1987).

An expression vector may be suitable for use in particular types of host cells and not others. One of ordinary skill in the art, however, can readily determine through routine experimentation whether a particular expression vector is suited for a given host cell. For example, the expression vector can be introduced into the host organism, which is then monitored for viability and expression of any genes contained in the vector.

The expression vector may also contain one or more selectable marker genes that, upon expression, confer one or more phenotypic traits useful for selecting or otherwise identifying host cells that carry the expression vector. Non-limiting examples of suitable selectable markers for eukaryotic cells include dihydrofolate reductase and neomycin resistance.

The vectors can be introduced into a host cell stably or transiently by variety of established techniques. For example, one method involves a calcium chloride treatment wherein the expression vector is introduced via a calcium precipitate. Other salts, for example calcium phosphate, may also be used following a similar procedure. In addition, electroporation (that is, the application of current to increase the permeability of cells to nucleic acids) may be used. Other transformation methods include microinjection, DEAE dextran mediated transformation, and heat shock in the presence of lithium acetate. Lipid complexes, liposomes, and dendrimers may also be employed to transfect the host cells.

Upon introduction of the heterologous sequence into a host cell, a variety of methods can be practiced to identify the host cells into which the subject vectors have been introduced. One exemplary selection method involves subculturing individual cells to form individual colonies, followed by testing for expression of the desired protein product. Another method entails selecting host cells containing the heterologous sequence based upon phenotypic traits conferred through the expression of selectable marker genes contained within the expression vector. Those of ordinary skill can identify genetically modified host cells using these or other methods available in the art.

For example, the introduction of various heterologous sequences of the disclosure into a host cell can be confirmed by methods such as PCR, Southern blot or Northern blot hybridization. For example, nucleic acids can be prepared from the resultant host cells, and the specific sequences of interest can be amplified by PCR using primers specific for the sequences of interest. The amplified product is subjected to agarose gel electrophoresis, polyacrylamide gel electrophoresis or capillary electrophoresis, followed by staining with ethidium bromide, SYBR Green solution or the like, or detection of DNA with a UV detection. Alternatively, nucleic acid probes specific for the sequences of interest can be employed in a hybridization reaction. The expression of a specific gene sequence can be ascertained by detecting the corresponding mRNA via reveres-transcription coupled PCR, Northern blot hybridization, or by immunoassays using antibodies reactive with the encoded gene product. Exemplary immunoassays include but are not limited to ELISA, radioimmunoassays, and sandwich immunoassays.

Furthermore, the introduction of various heterologous sequences of the disclosure into a host cell can be confirmed by the enzymatic activity of an enzyme that the heterologous sequence encodes. The enzyme can be assayed by a variety of methods known in the art. In general, the enzymatic activity can be ascertained by the formation of the product or conversion of a substrate of an enzymatic reaction that is under investigation. The reaction can take place in vitro or in vivo.

In some cases, the fusion polypeptide can be produced by expressing a vector in a cell under conditions suitable for protein expression. The suitable conditions for protein expression, including but not limited to factors such as incubation time, temperature, and medium, may be dependent on cell type and will be readily determined by one of ordinary skill in the art.

### Methods of Treatment

In one aspect, the invention provides methods for treating an autoimmune disease in a subject in need thereof, comprising administering to the subject a therapeutically effective amount of the fusion polypeptide of the present invention.

In some embodiments, the autoimmune disease is selected from psoriasis, rheumatoid arthritis, asthma, multiple sclerosis, type-1 diabetes, inflammatory bowel diseases, Crohn's disease, Hashimoto's thyreoiditis, autoimmune thyreoiditis, autoimmune myasthenia gravis, systemic lupus erythematosus, ulcerative colitis, atopic dermatitis, myocarditis and transplantation-related diseases such as graft-versus-host or host-versus graft reactions, or general organ tolerance issues. In some embodiments, the autoimmune disease is selected from asthma and atopic dermatitis.

In some embodiments of the method of the present invention, the subject is a human. In other embodiments, the subject can be an animal including but not limited to primates, domestic animals, farm animals, zoological garden animals or birds. For example, the animal can be mouse, a rat, a cat, a dog, a rabbit, a pig, a sheep, a horse, a bovine, a goat, a gerbil, a hamster, a guinea pig, a monkey or any other mammal.

In some embodiments, the therapeutically effective amount of the fusion polypeptide used in the method for treating the autoimmune disease is in a range from about 3 μg/kg to about 12.5 mg/kg. In some embodiments, the therapeutically effective amount of the fusion polypeptide is in a range from about 15 μg/kg to about 12.5 mg/kg. In some embodiments, the therapeutically effective amount of the fusion polypeptide is in a range from about 75 μg/kg to about 12.5 mg/kg. In some embodiments, the therapeutically effective amount of the fusion polypeptide is in a range from about 100 μg/kg to about 12.5 mg/kg. In some embodiments, the therapeutically effective amount of the fusion polypeptide is in a range from about 200 μg/kg to about 12.5 mg/kg. In some embodiments, the therapeutically effective amount of the fusion polypeptide is in a range from about 500 μg/kg to about 12.5 mg/kg. In some embodiments, the therapeutically effective amount of the fusion polypeptide is in a range from about 1 mg/kg to about 12.5 mg/kg. In some embodiments, the therapeutically effective amount of the fusion polypeptide is in a range from about 2 mg/kg to about 12.5 mg/kg. In some embodiments, the therapeutically effective amount of the fusion polypeptide is in a range from about 5 mg/kg to about 12.5 mg/kg. In some embodiments, the therapeutically effective amount of the fusion polypeptide is in a range from about 3 μg/kg to about 5 mg/kg. In some embodiments, the therapeutically effective amount of the fusion polypeptide is in a range from about 15 μg/kg to about 5 mg/kg. In some embodiments, the therapeutically effective amount of the fusion polypeptide is in a range from about 75 μg/kg to about 5 mg/kg. In some embodiments, the therapeutically effective amount of the fusion polypeptide is in a range from about 100 μg/kg to about 5 mg/kg. In some embodiments, the therapeutically effective amount of the fusion polypeptide is in a range from about 200 μg/kg to about 5 mg/kg. In some embodiments, the therapeutically effective amount of the fusion polypeptide is in a range from about 500 μg/kg to about 5 mg/kg. In some embodiments, the therapeutically effective amount of the fusion polypeptide is in a range from about 1 mg/kg to about 5 mg/kg. In some embodiments, the therapeutically effective amount of the fusion polypeptide is in a range from about 3 μg/kg to about 1 mg/kg. In some embodiments, the therapeutically effective amount of the fusion polypeptide is in a range from about 15 μg/kg to about 1 mg/kg. In some embodiments, the therapeutically effective amount of the fusion polypeptide is in a range from about 75 μg/kg to about 1 mg/kg. In some embodiments, the therapeutically effective amount of the fusion polypeptide is in a range from about 100 μg/kg to about 1 mg/kg. In some embodiments, the therapeutically effective amount of the fusion polypeptide is in a range from about 200 μg/kg to about 1 mg/kg. In some embodiments, the therapeutically effective amount of the fusion polypeptide is in a range from about 500 μg/kg to about 1 mg/kg. In some embodiments, the therapeutically effective amount of the fusion polypeptide is in a range from about 3 μg/kg to about 500 μg/kg. In some embodiments, the therapeutically effective amount of the fusion polypeptide is in a range from about 15 μg/kg to about 500 μg/kg. In some embodiments, the therapeutically effective amount of the fusion polypeptide is in a range from about 75 μg/kg to about 500 μg/kg. In some embodiments, the therapeutically effective amount of the fusion polypeptide is in a range from about 100 μg/kg to about 500 μg/kg. In some embodiments, the therapeutically effective amount of the fusion polypeptide is in a range from about 200 μg/kg to about 500 μg/kg.

In some embodiments, the method comprises administrating the therapeutically effective amount of the fusion polypeptide to the subject once daily, twice daily or three times daily.

In some embodiments, the method comprises administrating the therapeutically effective amount of the fusion polypeptide to the subject for at least 1 day. In some embodiments, the method comprises administrating the therapeutically effective amount of the fusion polypeptide to the subject for at least 2 days. In some embodiments, the method comprises administrating the therapeutically effective amount of the fusion polypeptide to the subject for at least 3 days. In some embodiments, the method comprises administrating the therapeutically effective amount of the fusion polypeptide to the subject for at least 4 days. In some embodiments, the method comprises administrating the therapeutically effective amount of the fusion polypeptide to the subject for at least 5 days. In some embodiments, the method comprises administrating the therapeutically effective amount of the fusion polypeptide to the subject for at least 6 days. In some embodiments, the method comprises administrating the therapeutically effective amount of the fusion polypeptide to the subject for at least a week. In some embodiments, the method comprises administrating the therapeutically effective amount of the fusion polypeptide to the subject for at least two weeks. In some embodiments, the method comprises administrating the therapeutically effective amount of the fusion polypeptide to the subject for at least a month. In some embodiments, the method comprises administrating the therapeutically effective amount of the fusion polypeptide to the subject for at least six months.

In some embodiments, at least one of the symptoms of the subject is improved as a result of administering the methods of the present invention. In some embodiments, at least one of the symptoms is improved within 3 hours as a result of administering the methods of the present invention. In some embodiments, at least one of the symptoms is improved within 4 hours as a result of administering the methods of the present invention. In some embodiments, at least one of the symptoms is improved within 5 hours as a result of administering the methods of the present invention. In some embodiments, at least one of the symptoms is improved within 6 hours as a result of administering the methods of the present invention. In some embodiments, at least one of the symptoms is improved within 7 hours as a result of administering the methods of the present invention. In some embodiments, at least one of the symptoms is improved within 8 hours as a result of administering the methods of the present invention. In some embodiments, at least one of the symptoms is improved within 9 hours as a result of administering the methods of the present invention. In some embodiments, at least one of the symptoms is improved within 10 hours as a result of administering the methods of the present invention. In some embodiments, at least one of the symptoms is improved within 11 hours as a result of administering the methods of the present invention. In some embodiments, at least one of the symptoms is improved within 12 hours as a result of administering the methods of the present invention. In some embodiments, at least one of the symptoms is improved within 1 day as a result of administering the methods of the present invention. In some embodiments, at least one of the symptoms is improved within 2 days as a result of administering the methods of the present invention. In some embodiments, at least one of the symptoms is improved within 3 days as a result of administering the methods of the present invention. In some embodiments, at least one of the symptoms is improved within 4 days as a result of administering the methods of the present invention. In some embodiments, at least one of the symptoms is improved within 5 days as a result of administering the methods of the present invention. In some embodiments, at least one of the symptoms is improved within 6 days as a result of administering the methods of the present invention. In some embodiments, at least one of the symptoms is improved within 1 week as a result of administering the methods of the present invention.

The fusion polypeptide of the present invention can be administered to the subject in any suitable route. In some embodiments, the routes of administration include, but are not limited to, oral, intravenous, rectal, aerosol, parenteral, ophthalmic, pulmonary, transmucosal, transdermal, vaginal, otic, nasal, and topical administration. In some embodiments, the parenteral delivery includes but is not limited to intramuscular, subcutaneous, intravenous, intrathecal, direct intraventricular, intraperitoneal, intralymphatic, and intranasal injections.

In another aspect, provided is use of the fusion polypeptide herein in preparing a medicament for treating an autoimmune disease in a subject in need thereof. In some embodiments, the autoimmune disease is selected from psoriasis, rheumatoid arthritis, asthma, multiple sclerosis, type-1 diabetes, inflammatory bowel diseases, Crohn's disease, Hashimoto's thyreoiditis, autoimmune thyreoiditis, autoimmune myasthenia gravis, systemic lupus erythematosus, ulcerative colitis, atopic dermatitis, myocarditis and transplantation-related diseases such as graft-versus-host or host-versus graft reactions, or general organ tolerance issues. In some embodiments, the autoimmune disease is selected from asthma and atopic dermatitis.

Pharmaceutical Composition

In another aspect, provided is a pharmaceutical composition comprising the fusion polypeptide as described above and a pharmaceutically acceptable excipient.

In some embodiments, the fusion polypeptide of the present invention is prepared into a pharmaceutical composition with one or more pharmaceutically acceptable excipients, carriers for treating autoimmune disease. The one or more pharmaceutically acceptable excipients, carriers include but are not limited to inert solid diluents and fillers, diluents, sterile aqueous solution and various organic solvents, permeation enhancers, solubilizers and adjuvants.

In some embodiment, the pharmaceutical composition for treating autoimmune disease may, for example, be in a form suitable for oral administration as a tablet, capsule, pill, powder, sustained release formulations, solution, suspension, for parenteral injection as a sterile solution, suspension or emulsion, for topical administration as a spray, ointment or cream. The pharmaceutical composition may be in unit dosage forms suitable for single administration In some embodiment, the pharmaceutical composition may include other medicinal or pharmaceutical agents, carriers, adjuvants, etc.

In some cases, the invention provides a method for treating autoimmune disease by using a pharmaceutical composition for injection containing the fusion polypeptide of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the fusion polypeptide of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

In some cases, the invention provides a method for treating autoimmune disease by using a pharmaceutical composition for oral administration containing the fusion polypeptide of the invention, and a pharmaceutical excipient suitable for oral administration.

In some cases, the invention provides a method for treating autoimmune disease by using a solid pharmaceutical composition for oral administration containing: (i) an effective amount of the fusion polypeptide of the invention; optionally (ii) an effective amount of a second agent; and (iii) a pharmaceutical excipient suitable for oral administration. In some embodiments, the composition further contains: (iv) an effective amount of a third agent.

In some cases, the invention provides a method for treating autoimmune disease by using a liquid pharmaceutical composition suitable for oral consumption. Pharmaceutical compositions of the invention suitable for oral administration for treating autoimmune disease can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation.

This invention provides a method for treating autoimmune disease by using an anhydrous pharmaceutical compositions and dosage forms comprising an active ingredient, since water can facilitate the degradation of some polypeptides. For example, water may be added (e.g., 5%) in the pharmaceutical arts as a means of simulating long-term storage in order to determine characteristics such as shelf-life or the stability of formulations over time. Anhydrous pharmaceutical compositions can be prepared using anhydrous or low moisture containing ingredients and low moisture or low humidity conditions. Pharmaceutical compositions which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

A fusion polypeptide can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the fusion polypeptide of the present invention and to minimize precipitation of the fusion polypeptide of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The pharmaceutical composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Kit

In another aspect, the invention provides a kit comprising the fusion polypeptide of the present invention and an instruction of administrating the fusion polypeptide for treating autoimmune disease. In some embodiments, the instruction further comprises the dosage of administrating the fusion polypeptide. In some embodiments, the dosage is 3 μg/kg-1.25 mg/kg. In some embodiments, the dosage is 75 μg/kg-1.25 mg/kg.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The examples and preparations provided below further illustrate and exemplify the fusion polypeptides of the present invention and methods of using and preparing thereof. It is to be understood that the scope of the present invention is not limited in any way by the scope of the following examples and preparations.

### Example 1: Preparation of the Fusion Polypeptide of the Present Application

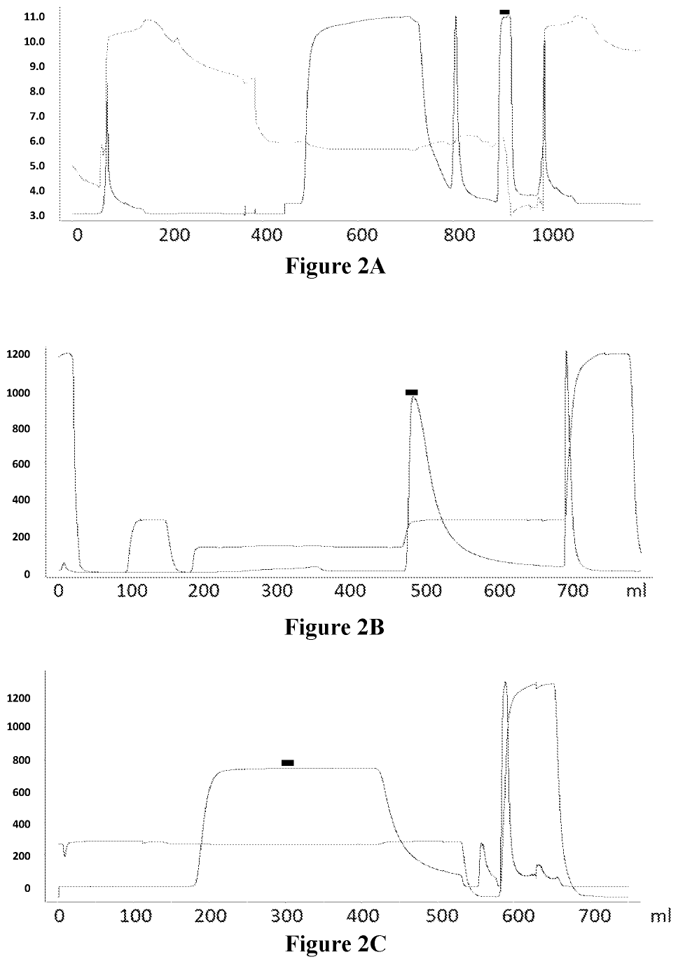

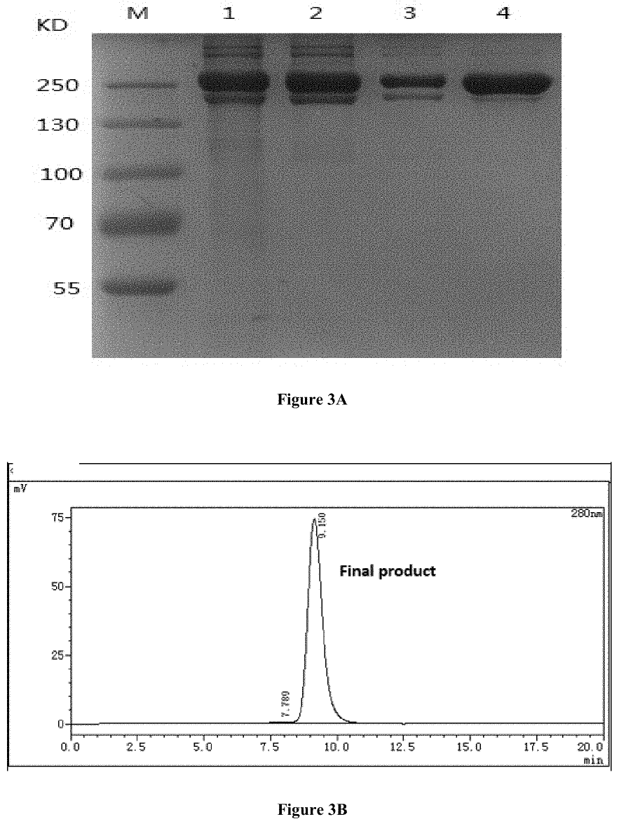

The fusion polypeptide with configuration of IL13Rα2-(GGGGS)₂-IL4Rα-(GGGGS)₂-hIgG4 Fc S228P (referred as IL13Rα2-IL4Rα-Fc hereinafter) was expressed in CHO cells. Then the cells were harvested, lysed and subject to high speed centrifuge. The supernatant was collected and further subject to three steps of purifications sequentially, including Mabselect SuRe LX affinity chromatography, Capto Q ImpRes ion exchange chromatography, and Capto adhere composite ion exchange chromatography. FIG. 2A, FIG. 2B and FIG. 2C respectively show the spectrums of the products after each of the three steps of purifications. FIG. 3A shows the products in SDS-PAGE gel, in which lane 1-4 of the gel were loaded with crude cell extract, purified product after Mabselect SuRe LX affinity chromatography, purified product after Capto Q ImpRes ion exchange chromatography and purified product after Capto adhere composite ion exchange chromatography, respectively. FIG. 3B shows the spectrum of the purified product after the three steps of purifications (black box indicating the target fusion polypeptide). As can be seen from FIG. 3A and FIG. 3B, the yield of the fusion polypeptide was up to 50% of the total protein, and the purity after the three steps of purifications was up to 98.5%.

### Example 2: Inhibition of Activities of hIL4 and hIL13 by the Fusion Polypeptide of the Present Application

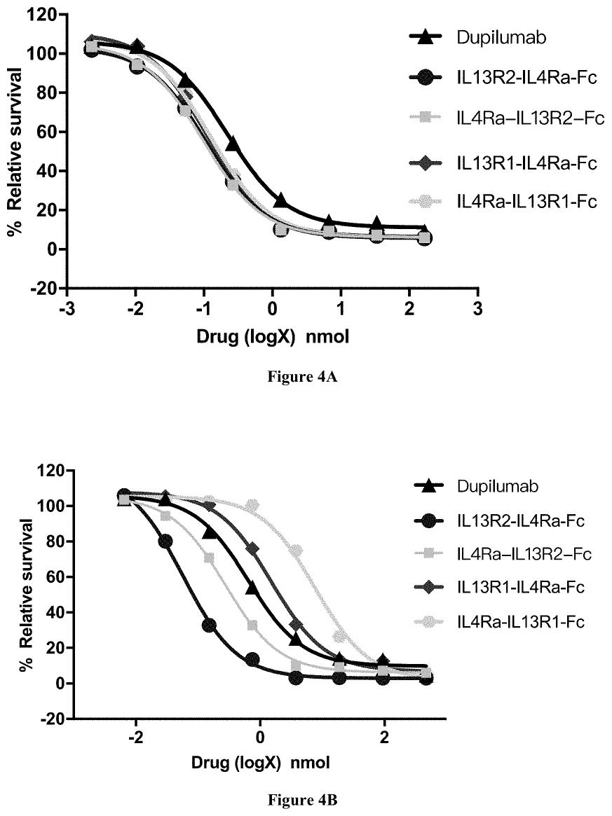

TF1 cells proliferate in response to human IL4 and IL13. In this study, Th1 cells were employed to explore the inhibition of the fusion polypeptide of the present application for IL4 and IL13. Dupilumab, an IL4Rα antibody and a dual inhibitor of IL4 and IL13 signaling, was used as the control to analyze the relative activity of fusion polypeptide in different structural configurations. Other than the IL13Rα2-IL4Rα-Fc prepared in Example 1 as described above, IL4Rα-IL13Rα2-Fc, IL13Rα1-IL4Rα-Fc and IL4Rα-IL13Rα1-Fc were also prepared and their inhibitions on IL4 and IL13 were analyzed in parallel with the IL13Rα2-IL4Rα-Fc of the present application. The result is as shown in FIG. 4A and FIG. 4B, as well as the Table 1 below. As can be seen from Table 1 and FIG. 4A, the IL13Rα2-IL4Rα-Fc of the present application showed stronger inhibition on IL4 as compared to Dupilumab, and comparative inhibition as IL4Rα-IL13Rα2-Fc, IL13Rα1-IL4Rα-Fc and IL4Rα-IL13Rα1-Fc. Meanwhile, as can be seen from Table 1 and FIG. 4B, IL13Rα2-IL4Rα-Fc of the present application showed significant stronger inhibition on IL13 as compared to Dupilumab and IL4Rα-IL13Rα2-Fc, IL13Rα1-IL4Rα-Fc and IL4Rα-IL13Rα1-Fc with other structural configurations.

TABLE 1
Inhibition of IL13 and IL4 by the fusion polypeptide

| | IC50 (nM) | | | |
|---|---|---|---|---|
| | Blocking | Relative | Blocking | Relative |
| Group | IL13 | activity | IL4 | activity |
| Dupilumab | 20.54 | 1 | 3.10 | 1 |
| IL13Ra2-IL4Rα-Fc | 1.02 | 20.1 | 1.10 | 2.8 |
| IL4Rα-IL13Rα2-Fc | 5.55 | 3.7 | 1.21 | 2.6 |
| IL13Rα1-IL4Rα-Fc | 46.1 | 0.4 | 1.24 | 2.5 |
| IL4Rα-IL13 Rα1-Fc | 201.4 | 0.1 | 1.36 | 2.3 |

### Example 3 Binding Affinities of the Fusion Polypeptide of the Present Application

The binding affinities of the fusion polypeptide with IL4 and IL13 were measured through surface plasmon resonance (SPR) binding analysis by using BIAcore 3000 instrument (GE Biosciences, Piscataway, N.J.).

Briefly, the fusion polypeptide was immobilized on the chip in HBS-EP buffer (10 mM HEPES, 15 Mm NaCl, 3.4 nM EDTA, 0.005% P20) at 25° C., and then binding of IL4 or IL13 with the fusion polypeptide was measured at the flow rate of 30 μL/minute for 3 minutes. Data was analyzed by using the Kinetics Wizard and the manual fitting programs in the BiaEvaluation Software V4.1.

The binding affinities of the fusion polypeptide with IL13 and IL4 are represented by using dissociation constant KD (KD=Ka/Kd), as shown in Table 2 below. As can be seen from Table 2, IL13Rα2-IL4Rα-Fc has higher affinities for both IL4 and IL13 as compared to the fusion polypeptides having other structural configurations, with KD of 1.68E-11 M for IL13 and KD of 7.41E-11 for IL4, respectively.

TABLE 2
Binding affinity of the fusion polypeptide with IL13 and IL4

| Sample | Ligand | ka (1/Ms) | kd (1/s) | KD (M) |
|---|---|---|---|---|
| IL13Rα2-IL4Rα-Fc | IL13 | 1.46E+06 | 2.45E−05 | 1.68E−11 |
| IL13Rα2-IL4Rα-Fc | IL4 | 4.72E+07 | 3.50E−03 | 7.41E−11 |
| IL4Rα-IL13Rα2-Fc | IL13 | 1.42E+06 | 6.03E−05 | 4.25E−11 |
| IL4Rα-IL13Rα2-Fc | IL4 | 4.12E+07 | 3.82E−03 | 9.28E−11 |
| IL13Rα1-IL4Rα-Fc | IL13 | 1.33E+05 | 7.52E−05 | 5.65E−10 |
| IL13Rα1-IL4Rα-Fc | IL4 | 3.10E+07 | 4.12E−03 | 1.33E−10 |
| IL4Rα-IL13Rα1-Fc | IL13 | 1.01E+05 | 9.85E−05 | 9.94E−10 |
| IL4Rα-IL13Rα1-Fc | IL4 | 2.90E+07 | 5.03E−03 | 1.73E−10 |

### Example 4 Mutual Interferences of IL13 and IL4 on Each Other's Binding with the Fusion Protein of the Present Application

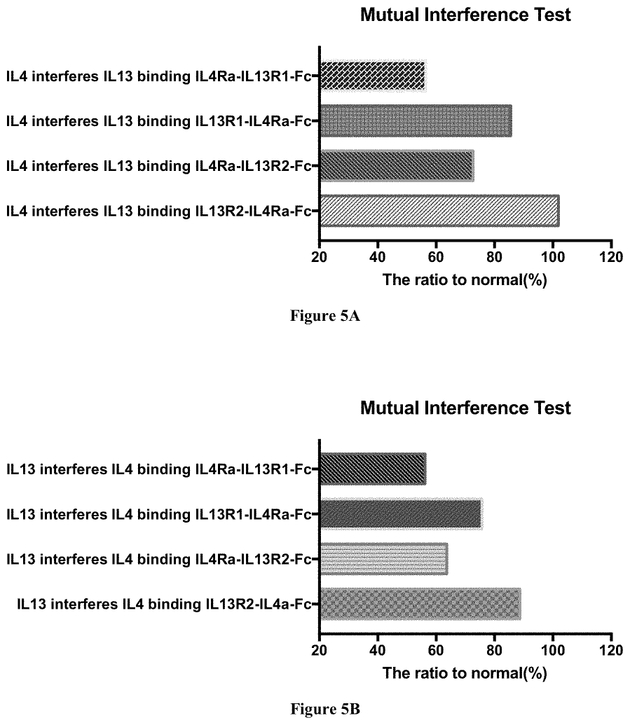

The fusion polypeptides with different structural configurations were coated on the bottom of the wells. To investigate interference of IL4 on the binding of IL13 with the fusion polypeptide, a fixed concentration of IL4 (1 ng/ml) and IL13 at gradient concentrations were mixed together and the mixture was added to the well and incubated with the fusion polypeptide for a period of time. IL13 at gradient concentrations without IL4 was used as the control. Then wells were washed and HRP conjugated IL13 antibody was added to the wells to detect IL13 bound to the polypeptide. The result is as show in FIG. 5A.

To investigate interference of IL13 on the binding of IL4 with the fusion polypeptide, a fixed concentration of IL13 (1 ng/ml) and IL4 at gradient concentrations were mixed together and the mixture was added to the well and incubated with the fusion polypeptide for a period of time. IL4 at gradient concentrations without IL13 was used as the control. Then wells were washed and HRP conjugated IL4 antibody was added to the wells to detect IL4 bound to the polypeptide. The result is as show in FIG. 5B.

As can be seen from FIGS. 5A and 5B, IL13 and IL4 displayed minimal interference on each other's binding with the IL13Rα2-IL4Rα-Fc of the present application.

While this disclosure has been described with an emphasis on preferred embodiments, it will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with the true scope and spirit of the disclosure being defined by the following claims.

Sequence Listing

| SEQ | | |
|---|---|---|
| ID | | |
| NO. | Sequences | cDesription |
| 1 | DTEIKVNPPQDFEIVDPGYLGYLYLQWQPPLSLDHFKECTVEYELKYRNIGS | IL13Rα2 |
| | ETWKTIITKNLHYKDGFDLNKGIEAKIHTLLPWQCTNGSEVQSSWAETTY | |
| | WISPQGIPETKVQDMDCVYYNWQYLLCSWKPGIGVLLDTNYNLFYWYEG | |
| | LDHALQCVDYIKADGQNIGCRFPYLEASDYKDFYICVNGSSENKPIRSSYFT | |
| | FQLQNIVKPLPPVYLTFTRESSCEIKLKWSIPLGPIPARCFDYEIEIREDDTTL | |
| | VTATVENETYTLKTTNETRQLCFVVRSKVNIYCSDDGIWSEWSDKQCWEG | |
| | EDLSKKTLLR (317 aa) | |
| 2 | MKVLQEPTCVSDYMSISTCEWKMNGPTNCSTELRLLYQLVFLLSEAHTCIP | IL4Rα |
| | ENNGGAGCVCHLLMDDVVSADNYTLDLWAGQQLLWKGSFKPSEHVKPR | |
| | APGNLTVHTNVSDTLLLTWSNPYPPDNYLYNHLTYAVNIWSENDPADFRI | |
| | YNVTYLEPSLRIAASTLKSGISYRARVRAWAQCYNTTWSEWSPSTKWHNS | |
| | YREPFEQH (207 aa) | |
| 3 | GGGGAAPTETQPPVTNLSVSVENLCTVIWTWNPPEGASSNCSLWYFSHFG | IL13Rα1 |
| | DKQDKKIAPETRRSIEVPLNERICLQVGSQCSTNESEKPSILVEKCISPPEGDP | |
| | ESAVTELQCIWHNLSYMKCSWLPGRNTSPDTNYTLYYWHRSLEKIHQCEN | |
| | IFREGQYFGCSFDLTKVKDSSFEQHSVQIMVKDNAGKIKPSFNIVPLTSRVK | |
| | PDPPHIKNLSFHNDDLYVQWENPQNFISRCLFYEVEVNNSQTETHNVFYVQ | |
| | EAKCENPEFERNVENTSCFMVPGVLPDTLNTVRIRVKTNKLCYEDDKLWS | |
| | NWSQEMSIGKKRNST (322 aa) | |
| 4 | HKCDITLQEIIKTLNSLTEQKTLCTELTVTDIFAASKNTTEKETFCRAATVLR | IL4 |
| | QFYSHHEKDTRCLGATAQQFHRHKQLIRFLKRLDRNLWGLAGLNSCPVKE | |
| | ANQSTLENFLERLKTIMREKYSKCSS (129 aa) | |
| 5 | PVPPSTALRELIEELVNITQNQKAPLCNGSMVWSINLTAGMYCAALESLINV | IL13 |
| | SGCSAIEKTQRMLSGFCPHKVSAGQFSSLHVRDTKIEVAQFVKDLLLHLKK | |
| | LFREGRFN (111 aa) | |
| 6 | EPKSCDKTHTCPPCPAPELLGGPSVFLFPPKPKDTLMISRTPEVTCVVVDVS | IgG1 |
| | HEDPEVKFNWYVDGVEVHNAKTKPREEQYNSTYRVVSVLTVLHQDWLN | Fc |
| | GKEYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRWQ | |
| | QGNVFSCSVMHEALHNHYTQKSLSLSPGK (232 aa) | |
| 7 | EPKSCDKTHTCPPCPAPEAEGAPSVFLFPPKPKDTLMISRTPEVTCVVVDVS | IgG1 |
| | HEDPEVKFNWYVDGVEVHNAKTKPREEQYNSTYRVVSVLTVLHQDWLNG | Fc |
| | KEYKCKVSNKALPSSIEKTISKAKGQPREPQVYTLPPSRDELTKNQVSLTCL | mu |
| | VKGFYPSDIAVEWESNGQPENNYKTTPPVLDSDGSFFLYSKLTVDKSRWQQ | (AEASS) |
| | GNVFSCSVMHEALHNHYTQKSLSLSPGK (232 aa) | |
| 8 | ERKCCVECPPCPAPPVAGPSVFLFPPKPKDTLMISRTPEVTCVVVDVSHEDP | IgG2 |
| | EVQFNWYVDGMEVHNAKTKPREEQFNSTFRVVSVLTVVHQDWLNGKEY | Fc |
| | KCKVSNKGLPAPIEKTISKTKGQPREPQVYTLPPSREEMTKNQVSLTCLVK | |
| | GFYPSDIAVEWESNGQPENNYKTTPPMLDSDGSFFLYSKLTVDKSRWQQG | |
| | NVFSCSVMHEALHNHYTQKSLSLSPGK (228 aa) | |
| 9 | ESKYGPPCPPCPAPEFLGGPSVFLFPPKPKDTLMISRTPEVTCVVVDVSQEDP | IgG4 |
| | EVQFNWYVDGVEVHNAKTKPREEQFNSTYRVVSVLTVLHQDWLNGKEY | Fc |
| | KCKVSNKGLPSSIEKTISKAKGQPREPQVYTLPPSQEEMTKNQVSLTCLVK | S228P |
| | GFYPSDIAVEWESNGQPENNYKTTPPVLDSDGSFFLYSRLTVDKSRWQEGN | |
| | VFSCSVMHEALHNHYTQKSLSLSLGK (229 aa) | |
| 10 | DAHKSEVAHRFKDLGEENFKALVLIAFAQYLQQCPFEDHVKLVNEVTEFA | HSA |
| | KTCVADESAENCDKSLHTLFGDKLCTVATLRETYGEMADCCAKQEPERNE | |
| | CFLQHKDDNPNLPRLVRPEVDVMCTAFHDNEETFLKKYLYEIARRHPYFY | |
| | APELLFFAKRYKAAFTECCQAADKAACLLPKLDELRDEGKASSAKQRLKC | |
| | ASLQKFGERAFKAWAVARLSQRFPKAEFAEVSKLVTDLTKVHTECCHGDL | |
| | LECADDRADLAKYICENQDSISSKLKECCEKPLLEKSHCIAEVENDEMPAD | |
| | LPSLAADFVESKDVCKNYAEAKDVFLGMFLYEYARRHPDYSVVLLLRLAK | |
| | TYETTLEKCCAAADPHECYAKVFDEFKPLVEEPQNLIKQNCELFEQLGEYK | |
| | FQNALLVRYTKKVPQVSTPTLVEVSRNLGKVGSKCCKHPEAKRMPCAEDY | |
| | LSVVLNQLCVLHEKTPVSDRVTKCCTESLVNRRPCFSALEVDETYVPKEFN | |
| | AETFTFHADICTLSEKERQIKKQTALVELVKHKPKATKEQLKAVMDDFAAF | |
| | VEKCCKADDKETCFAEEGKKLVAASQAALGL (585 aa) | |
| 11 | DTEIKVNPPQDFEIVDPGYLGYLYLQWQPPLSLDHFKECTVEYELKYRNIGS | IL13Rα2- |
| | ETWKTIITKNLHYKDGFDLNKGIEAKIHTLLPWQCTNGSEVQSSWAETTY | IL4Rα- |
| | WISPQGIPETKVQDMDCVYYNWQYLLCSWKPGIGVLLDTNYNLFYWYEG | hIg |
| | LDHALQCVDYIKADGQNIGCRFPYLEASDYKDFYICVNGSSENKPIRSSYFT | G4 |
| | FQLQNIVKPLPPVYLTFTRESSCEIKLKWSIPLGPIPARCFDYEIEIREDDTTL | Fc |
| | VTATVENETYTLKTTNETRQLCFVVRSKVNIYCSDDGIWSEWSDKQCWEG | S228P |
| | EDLSKKTLLRGGGGSGGGGSMKVLQEPTCVSDYMSISTCEWKMNGPTNCS | |
| | TELRLLYQLVFLLSEAHTCIPENNGGAGCVCHLLMDDVVSADNYTLDLWA | |
| | GQQLLWKGSFKPSEHVKPRAPGNLTVHTNVSDTLLLTWSNPYPPDNYLYN | |
| | HLTYAVNIWSENDPADFRIYNVTYLEPSLRIAASTLKSGISYRARVRAWAQ | |
| | CYNTTWSEWSPSTKWHNSYREPFEQHGGGGSGGGGSESKYGPPCPPCPAP | |
| | EFLGGPSVFLFPPKPKDTLMISRTPEVTCVVVDVSQEDPEVQFNWYVDGVE | |
| | VHNAKTKPREEQFNSTYRVVSVLTVLHQDWLNGKEYKCKVSNKGLPSSIE | |
| | KTISKAKGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMHEALHN | |
| | HYTQKSLSLSLGK | |

1. A fusion polypeptide comprising a structure of formula I arranged from amino terminus to carboxyl terminus as:
X1-(S1)-X2-(S2)-X3
wherein each of S1 and S2 is independently a spacer, and each of X1, X2 and X3 is independently selected from IL13Rα2, IL4Rα and a regulatory component, with the proviso that the IL13Rα2 is closer to the amino terminus than the IL4Rα.

2. (canceled)

3. The fusion polypeptide of claim 1, wherein the IL13Rα2 comprises an amino acid sequence having at least 90% identity with SEQ ID NO: 1.

4. (canceled)

5. The fusion polypeptide of claim 1, wherein the IL13Rα2 comprises an amino acid sequence of SEQ ID NO: 1.

6. (canceled)

7. The fusion polypeptide of claim 1, wherein the IL13Rα2 comprises a non-natural amino acid as compared to SEQ ID NO: 1.

8. (canceled)

9. The fusion polypeptide of claim 1, wherein the IL13Rα2 comprises a modification as compared to SEQ ID NO: 1.

10. (canceled)

11. The fusion polypeptide of claim 9, wherein the modification is selected from the group consisting of pegylation, amidation, glycosylation, acylation, sulfation, phosphorylation, acetylation, cyclization, and any combination thereof.

12. (canceled)

13. The fusion polypeptide of claim 1, wherein the IL4Rα comprises an amino acid sequence having at least 90% identity with SEQ ID NO: 2.

14-16. (canceled)

17. The fusion polypeptide of claim 1, wherein the IL4Rα comprises a non-natural amino acid as compared to SEQ ID NO: 2.

18. (canceled)

19. The fusion polypeptide of claim 1, wherein the IL4Rα comprises a modification as compared to SEQ ID NO: 2.

20. (canceled)

21. The fusion polypeptide of claim 19, wherein the modification is selected from the group consisting of pegylation, amidation, glycosylation, acylation, sulfation, phosphorylation, acetylation, cyclization, and any combination thereof.

22. The fusion polypeptide of claim 1, wherein the regulatory component is selected from a group consisting of Fc domain, serum albumin, CTP, ELP, XTEN, and any fragment thereof.

23. (canceled)

24. (canceled)

25. The fusion polypeptide of claim 1, wherein the regulatory component comprises an Fc domain, and wherein the Fc domain comprises an amino acid of SEQ ID NO: 6, SEQ ID NO: 7, SEQ ID NO: 8, or SEQ ID NO: 9.

26-31. (canceled)

32. The fusion polypeptide of claim 31, wherein the regulatory component prolongs half-life, stability, or both of the fusion polypeptide.

33-37. (canceled)

38. The fusion polypeptide of claim 1, wherein the spacer is selected from a group consisting of (GS)ₙ (SEQ ID NO: 12), (GGS)ₙ (SEQ ID NO: 13), (GGGS)ₙ (SEQ ID NO: 14), (GGSG)ₙ (SEQ ID NO: 15), (GGSGG)ₙ (SEQ ID NO: 16), (GGGGS)ₙ (SEQ ID NO: 17) and null, wherein n is 1, 2, 3, 4, 5, 6, 7, 8, 9, or 10.

39. (canceled)

40. A pharmaceutical composition comprising the fusion polypeptide of claim 1, and a pharmaceutically acceptable excipient.

41. (canceled)

42. An isolated polynucleotide encoding the fusion polypeptide of claim 1.

43. A host cell expressing the fusion polypeptide of claim 1.

44. A kit comprising the fusion polypeptide of claim 1 and an instruction for using the kit.

45. A method for treating an autoimmune disease comprising administrating a therapeutically effective amount of the fusion polypeptide of claim 1 to a subject in need thereof.

46. The method of claim 45, wherein the autoimmune disease is selected from psoriasis, rheumatoid arthritis, asthma, multiple sclerosis, type-1 diabetes, inflammatory bowel diseases, Crohn's disease, Hashimoto's thyreoiditis, autoimmune thyreoiditis, autoimmune myasthenia gravis, systemic lupus erythematosus, ulcerative colitis, atopic dermatitis, myocarditis and transplantation-related diseases such as graft-versus-host or host-versus graft reactions, or general organ tolerance issues.

47-50. (canceled)
